(19) United States
(12) Patent Application Publication
(10) Pub. No.: US 2024/0181002 A1
(43) Pub. Date: Jun. 6, 2024

(54) COMPOSITION FOR ANTI-OBESITY COMPRISING GREEN TEA PEPTIDE COMPOSITION

(71) Applicant: AMOREPACIFIC CORPORATION, Seoul (KR)

(72) Inventors: Hyun Woo JEONG, Yongin-si (KR); Jinoh CHUNG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/515,599

(22) Filed: Nov. 21, 2023

(30) Foreign Application Priority Data
Dec. 2, 2022 (KR) 10-2022-0166719

(51) Int. Cl. A61K 38/16; A61P 3/04

(52) U.S. Cl. CPC A61K 38/168; A61P 3/04; A61K 2236/19; A61K 2236/333; A61K 2236/37

(57) ABSTRACT

The present disclosure relates to a composition for anti-obesity including a green tea peptide composition. Specifically, the green tea peptide composition according to the one aspect of the present disclosure exhibits anti-obesity efficacy through excellent lipid metabolism activation and can be applied to various health functional food compositions and pharmaceutical compositions.

### CROSS-REFERENCE TO RELATED APPLICATION

The present application claims priority to Korean Patent Application No. 10-2022-0166719, filed on Dec. 2, 2022, the entire contents of which is incorporated herein for all purposes by this reference.

The instant application contains a Sequence Listing which has been submitted electronically in XML format and is hereby incorporated by reference in its entirety. Said XML copy, created on Oct. 19, 2023 is named 3549-135Seq.XML and is 8,192 bytes in size.

### BACKGROUND OF THE INVENTION

### Technical Field

The present disclosure relates to a composition for anti-obesity including a green tea peptide composition.

### Background Art

The tea plant is one of 82 species classified in the genus Camellia and is currently cultivated in more than 50 countries, mainly in Asia, but also in Africa, South America, and Oceania. According to the processing method of tea leaves, the types of tea are broadly classified into unfermented tea, semi-fermented tea, fermented tea, and post-fermented tea. Among them, unfermented tea is made by inactivating the polyphenol oxidase contained in the tea plant by heat treatment, and contains more polyphenol types such as flavonol, flavanone, and flavonoid than other teas, exhibiting strong antioxidant power, and these substances account for approximately 30% of the dry weight of tea.

As the pharmacological mechanisms of various components in green tea have been gradually revealed, its value has been recognized by the general public, and in particular, the effects of antioxidant, anticancer, cholesterol-lowering, anti-aging, heavy metal detoxification, tooth decay prevention, and bad breath removal by polyphenols, the main components of green tea, have been proven, thus attracting wide attention.

Peptides are the most oxidation-stable substances in plants and are expected to have high skin efficacy due to their simple structure. In plants, the peptides act as a signal transmitting material and are known to be particularly involved in plant growth, differentiation, and response to external stimuli.

Meanwhile, obesity is an excessive accumulation of body fat due to an imbalance between energy intake and consumption, resulting in an increase in the number and size of fat cells. Energy in the body is stored in the form of triglycerides in fat cells, and when the energy source is depleted, the stored fat is decomposed into free fatty acids and glycerol to be used as an energy source, but excessive energy intake promotes the differentiation of fat cells and increases the amount of stored fat in the body, which becomes a direct cause of obesity.

Obesity is a risk factor for the development of various diseases, as well as changes in body shape due to the accumulation of fat in the internal organs and abdomen. Excessive accumulation of visceral fat can lead to problems with sugar metabolism, abnormalities in hormone secretion, cytokine secretion, and other symptoms. Obesity causes an increase in triglycerides, LDL-cholesterol, and a decrease in HDL-cholesterol, which leads to abnormalities in fat metabolism, a decrease in insulin receptors in tissues, and a decrease in insulin sensitivity, which inhibits the transport of glucose into cells, leading to high blood sugar and diabetes. In addition, obesity has been known to be strongly associated with the development of metabolic diseases such as hyperlipidemia, cardiovascular disease, cancer, respiratory disorders, stroke, and osteoarthritis.

Existing anti-obesity drugs such as orlistat and sibutramine are known to have serious side effects, such as vomiting, constipation, gastrointestinal disorders, and cardiovascular disease, leading to ongoing efforts to develop effective and safer substances. Retinol, vitamin E, vitamin U, chinese pepper tree extract, and the like have been reported as materials that inhibit the differentiation of fat cells, and research is being actively conducted to develop anti-obesity drugs coming from natural sources that can be safely and sustainably ingested.

### SUMMARY OF THE INVENTION

The present disclosure aims to provide a composition including a green tea peptide that has a novel amino acid sequence isolated and purified by culturing a green tea protein and a specific plant-based lactic acid bacterium as an active ingredient, which exhibits an excellent anti-obesity effect.

To achieve the above object, an embodiment of the present invention provides a composition for anti-obesity, including a green tea peptide composition as an active ingredient.

The green tea peptide composition according to the present disclosure exhibits anti-obesity efficacy through an excellent lipid metabolism activation and can be applied to various health functional food compositions, and pharmaceutical compositions.

### BRIEF DESCRIPTION OF THE DRAWINGS

FIG. 1 schematically illustrates a manufacturing process for a green tea peptide according to an embodiment of the present invention.

FIGS. 2A to 2D illustrate results of confirming an effect of inhibition of fat synthesis in a fat cell by a concentration of a green tea peptide composition (GTP) according to Experimental example 1 (*** P<0.001 vs. (−), ** P<0.01 vs. (−), *P<0.05 vs. (−)).

FIGS. 3A to 3D illustrate results of confirming an effect of promoting fat oxidation in a fat cell by a concentration of the green tea peptide composition (GTP) according to Experimental example 1 (*** P<0.001 vs. (−), ** P<0.01 vs. (−), *P<0.05 vs. (−)).

FIGS. 4A to 4E illustrate results of confirming an effect of promoting mitochondrial biosynthesis-related gene expression in a fat cell by a concentration of the green tea peptide composition (GTP) according to Experimental example 2 (*** P<0.001 vs. (−), ** P<0.01 vs. (−), *P<0.05 vs. (−)).

FIG. 5 illustrates results of confirming an effect of promoting mitochondrial biosynthesis in a fat cell by a concentration of the green tea peptide composition (GTP) according to Experimental example 2 (*** P<0.01 vs. (−), *P<0.05 vs. (−)).

FIG. 6 illustrates results of confirming an effect of inhibiting lipid accumulation in a fat cell of the green tea peptide composition (GTP) according to Experimental example 3 (*** P<0.001 vs. (−), ** P<0.01 vs. (−), *P<0.05 vs. (−)).

FIGS. 7A to 7D illustrate results of confirming an effect of inhibiting the inflammatory response in a fat cell of the green tea peptide composition (GTP) according to Experimental example 4 (*** P<0.001 vs. FFA, ** P<0.01 vs. FFA, *P<0.05 vs. FFA).

FIG. 8 illustrates results of a comparison of an effect of inhibiting fat accumulation in a fat cell of the green tea peptides according to a processing method according to Experimental example 5 (*** P<0.001 vs. (−), *P<0.05 vs. (−)).

FIG. 9 illustrates a result of comparing the similarity between Lacticaseibacillus paracasei (L. paracasei) and Lactiplantibacillus plantarum.

FIGS. 10A and 10B illustrate results of analyzing a molecular weight of a green tea proteolytic product by each of Lacticaseibacillus paracasei (L. paracasei) and Lactiplantibacillus plantarum.

FIG. 11 illustrates results of comparing an effect of inhibiting fat accumulation by molecular weight fraction of the green tea peptide composition (GTP) according to Experimental example 6 (*** P<0.001 vs. (−)).

### DETAILED DESCRIPTION OF THE INVENTION

Hereinafter, the present invention will be described in detail.

In the present specification, the term “green tea (tea; Camellia Sinensis)” refers to an evergreen broad-leaved shrub of the family Teaaceae, the tea from the dried leaves of which has been used in a variety of ways. In particular, the green tea is known to exhibit the effects of antioxidant, anticancer, and cardiovascular effects, reducing lipids in the blood and promoting blood circulation. The green tea includes one or more of those selected from the group consisting of tea plant leaves, flowers, stems, fruits, roots, stems, and heartwood of roots, and may preferably be leaves.

In the present specification, the term “active ingredient” means an ingredient that may exhibit the intended activity alone or in combination with a carrier that is not itself active.

In the present disclosure, the term “anti-obesity” means reduction of body fat, inhibition of body fat accumulation, and/or reduction of body weight. Therefore, the “anti-obesity” means the prevention, amelioration, and treatment of obesity, and further includes the reduction of body weight/body fat for cosmetic or health purposes (a.k.a. for the purpose of dieting), even though the body weight is not classified as obesity or excess weight.

In the present specification, the term “obesity” means a state in which there is an abnormal increase in fat tissue, whether due to genetic or environmental factors, and includes, based on the classification of body mass index (BMI), severe obesity (BMI of 30.0 or more), obesity (BMI of 25-30), and excess weight (BMI of 23-25).

In one aspect, the present invention may relate to an anti-obesity composition including a green tea peptide composition as an active ingredient.

In another aspect, the present invention may relate to a method of preventing, ameliorating, or treating obesity, including a step of administering an active amount of a green tea peptide composition to a subject in need thereof.

In still another aspect, the present invention may relate to a use of a green tea peptide composition for preparing an anti-obesity composition.

In an embodiment, the green tea peptide composition may include one or more species of green tea peptides including an amino acid sequence selected from the group consisting of SEQ ID NO: 1 to SEQ ID NO: 7. Specifically, the green tea peptide may include an amino acid sequence selected from the group consisting of AYKRRKGKFA (SEQ ID NO: 1), FFFFFFFFFFFFFFFYL (SEQ ID NO: 2), ISKIWNSEVPETEVKNEAESP (SEQ ID NO: 3), PFFCEKMMETN (SEQ ID NO: 4), RFLHERMAYYH (SEQ ID NO: 5), RNLNRLQRLLSMKQEYSPRNHLGSRWREY (SEQ ID NO: 6), and TTSSRKKEKPRRFWNNHEEVFLITTK (SEQ ID NO: 7).

In an embodiment, 60% (w/w) or more, 65% (w/w) or more, 70% (w/w) or more, 75% (w/w) or more, 80% (w/w) or more, 85% (w/w) or more, 90% (w/w) or more, or 95% (w/w) or more of the green tea peptide composition may be composed of one or more species of green tea peptides.

In an embodiment, the green tea peptide composition may be obtained by fermenting a green tea protein with a plant-based lactic acid bacterium.

In an embodiment, the fermentation may be performed at a condition of pH 5 to 8. Specifically, the fermentation may be performed at pH 5 or more, pH 5.2 or more, pH 5.4 or more, pH 5.6 or more, pH 5.8 or more, pH 6 or more, pH 6.2 or more, pH 6.4 or more, pH 6.6 or more, pH 6.8 or more, pH 7 or more, pH 7.2 or more, pH 7.4 or more, pH 7.6 or more or pH 7.8 or more. In addition, the fermentation may be performed at pH 8 or less, pH 7.8 or less, pH 7.6 or less, pH 7.4 or less, pH 7.2 or less, pH 7 or less, pH 6.8 or less, pH 6.6 or less, pH 6.4 or less, pH 6.2 or less, pH 6 or less, pH 5.8 or less, pH 5.6 or less, pH 5.4 or less or pH 5.2 or less. Preferably, the fermentation may be performed at pH 6.8.

In an embodiment, the fermentation may be performed at 25 to 45° C. Specifically, the fermentation may be performed at 25° C. or more, 27° C. or more, 29° C. or more, 31° ° C. or more, 33° C. or more, 35° C. or more, 37° C. or more, 39ºC or more, 41° C. or more or 43° C. or more. In addition, the fermentation may be performed at 45° C. or less, 43° C. or less, 41° C. or less, 39ºC or less, 37° C. or less, 35° C. or less, 33° C. or less, 31ºC or less, 29° C. or less or 27° ° C. or less. Preferably, the fermentation may be performed at 37ºC.

In an embodiment, the fermentation may be performed for 24 to 72 hours. Specifically, the fermentation may be performed for 24 hours or more, 26 hours or more, 28 hours or more, 30 hours or more, 32 hours or more, 34 hours or more, 36 hours or more, 38 hours or more, 40 hours or more, 42 hours or more, 44 hours or more, 46 hours or more, 48 hours or more, 50 hours or more, 52 hours or more, 54 hours or more, 56 hours or more, 58 hours or more, 60 hours or more, 62 hours or more, 64 hours or more, 66 hours or more, 68 hours or more or 70 hours or more. In addition, the fermentation may be performed for 72 hours or less, 70 hours or less, 68 hours or less, 66 hours or less, 64 hours or less, 62 hours or less, 60 hours or less, 58 hours or less, 56 hours or less, 54 hours or less, 52 hours or less, 50 hours or less, 48 hours or less, 46 hours or less, 44 hours or less, 42 hours or less, 40 hours or less, 38 hours or less, 36 hours or less, 34 hours or less, 32 hours or less, 30 hours or less, 28 hours or less or 26 hours or less. Preferably, the fermentation may be performed for 48 hours.

In an embodiment, the plant-based lactic acid bacterium may be Lactiplantibacillus plantarum. More specifically, the plant-based lactic acid bacterium may be Lactiplantibacillus plantarum APsulloc 331261 (Korean Culture Center of Microorganisms, accession number KCCM11179P, accession date 20110328).

In an embodiment, the green tea protein may be obtained from a residue of a primary extract that is extracted from green tea with anhydrous or hydrous C1-C6 low carbon alcohol.

In an embodiment, the concentration of alcohol in the hydrous C1-C6 low carbon alcohol may be 20 to 80% (v/v). Specifically, the concentration of alcohol in the hydrous C1-C6 low carbon alcohol may be 20% (v/v) or more, 22% (v/v) or more, 24% (v/v) or more, 26% (v/v) or more, 28% (v/v) or more, 30% (v/v) or more, 32% (v/v) or more, 34% (v/v) or more, 36% (v/v) or more, 38% (v/v) or more, 40% (v/v) or more, 42% (v/v) or more, 44% (v/v) or more, 46% (v/v) or more, 48% (v/v) or more, 50% (v/v) or more, 52% (v/v) or more, 54% (v/v) or more, 56% (v/v) or more, 58% (v/v) or more, 60% (v/v) or more, 62% (v/v) or more, 64% (v/v) or more, 66% (v/v) or more, 68% (v/v) or more, 70% (v/v) or more, 72% (v/v) or more, 74% (v/v) or more, 76% (v/v) or more or 78% (v/v) or more, also may be 80% (v/v) or less, 78% (v/v) or less, 76% (v/v) or less, 74% (v/v) or less, 72% (v/v) or less, 70% (v/v) or less, 68% (v/v) or less, 66% (v/v) or less, 64% (v/v) or less, 62% (v/v) or less, 60% (v/v) or less, 58% (v/v) or less, 56% (v/v) or less, 54% (v/v) or less, 52% (v/v) or less, 50% (v/v) or less, 48% (v/v) or less, 46% (v/v) or less, 44% (v/v) or less, 42% (v/v) or less, 40% (v/v) or less, 38% (v/v) or less, 36% (v/v) or less, 34% (v/v) or less, 32% (v/v) or less, 30% (v/v) or less, 28% (v/v) or less, 26% (v/v) or less, 24% (v/v) or less or 22% (v/v) or less.

In an embodiment, the hydrous C1-C6 low carbon alcohol may be a 20 to 80% (v/v) ethanol aqueous solution. Specifically, the hydrous C1-C6 low carbon alcohol may be a 20% (v/v) ethanol aqueous solution, a 25% (v/v) ethanol aqueous solution, a 30% (v/v) ethanol aqueous solution, a 35% (v/v) ethanol aqueous solution, a 40% (v/v) ethanol aqueous solution, a 41% (v/v) ethanol aqueous solution, a 42% (v/v) ethanol aqueous solution, a 43% (v/v) ethanol aqueous solution, a 44% (v/v) ethanol aqueous solution, a 45% (v/v) ethanol aqueous solution, a 46% (v/v) ethanol aqueous solution, a 47% (v/v) ethanol aqueous solution, a 48% (v/v) ethanol aqueous solution, a 49% (v/v) ethanol aqueous solution, a 50% (v/v) ethanol aqueous solution, a 51% (v/v) ethanol aqueous solution, a 52% (v/v) ethanol aqueous solution, a 53% (v/v) ethanol aqueous solution, a 54% (v/v) ethanol aqueous solution, a 55% (v/v) ethanol aqueous solution, a 56% (v/v) ethanol aqueous solution, a 57% (v/v) ethanol aqueous solution, a 58% (v/v) ethanol aqueous solution, a 59% (v/v) ethanol aqueous solution, a 60% (v/v) ethanol aqueous solution, a 65% (v/v) ethanol aqueous solution, a 70% (v/v) ethanol aqueous solution, a 75% (v/v) ethanol aqueous solution or a 80% (v/v) ethanol aqueous solution.

In an embodiment, the green tea protein may be obtained from a residue of a secondary extract which is hydrothermally extracted from the residue of the primary extract.

In an embodiment, the green tea protein may be obtained from the residue of the secondary extract through the processes of alkaline extraction, filtration and acid precipitation.

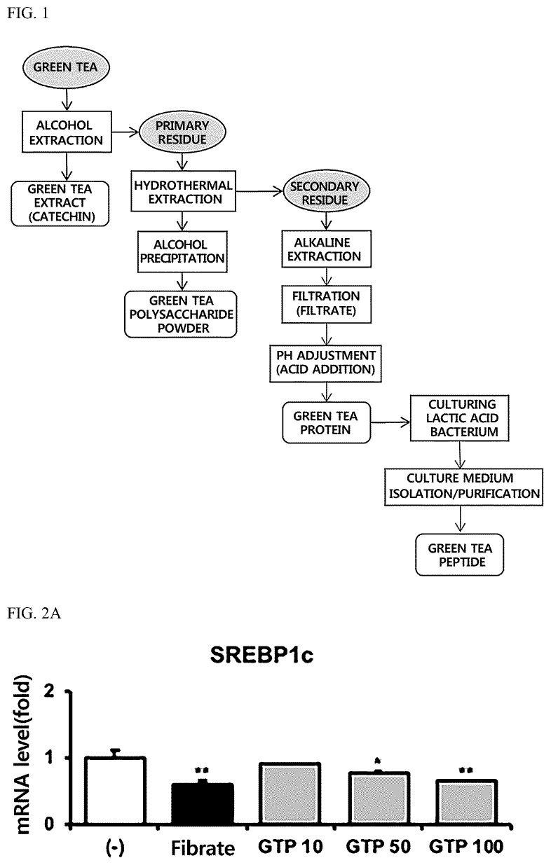

As illustrated in FIG. 1, the green tea peptide composition according to an embodiment of the present invention can be obtained by hydrothermal secondary extraction of the residue remaining after the primary extraction with green tea as a main alcohol, and by culturing the green tea protein obtained by alkaline extraction, filtration, and acid precipitation of the residue remaining after the secondary extraction with lactic acid bacterium.

In an embodiment, the green tea peptide composition may inhibit lipid synthesis in the fat cell.

In an embodiment, the green tea peptide composition may reduce the expression of one or more of sterol regulatory element-binding protein-1c (SREBP1c), acetyl-CoA carboxylase (ACC), fatty acid synthase (FAS), and stearoyl-CoA desaturase-1 (SCD1).

Specifically, the green tea peptide composition may inhibit lipid synthesis in the fat cell by reducing the expression of one or more of SREBP1c, ACC, FAS, and SCD1.

In an embodiment, the green tea peptide composition may promote fat oxidation in the fat cell.

In an embodiment, the green tea peptide composition may increase the expression of one or more of acyl-CoA oxidase (ACO), carnitine-palmitoyl transferase (CPT), medium-chain acyl-CoA dehydrogenase (mCAD), and peroxisome proliferator-activated receptor alpha (PPARa).

Specifically, the green tea peptide composition may promote fat oxidation in the fat cell by increasing the expression of one or more of ACO, CPT, mCAD, and PPARa.

In an embodiment, the green tea peptide composition may promote mitochondrial biosynthesis in the fat cell.

In an embodiment, the green tea peptide composition may increase the expression of one or more of mitochondrial transcription factor A (TFAM), NADH dehydrogenase [ubiquinone] 1 alpha subcomplex subunit 9 (NDUFA9), cytochrome c oxidase subunit 4 (COX4), adenosine triphosphate synthase, mitochondrial F1 complex, subunit alpha (ATP5a), and mitochondrial uncoupling protein 2 (UCP2).

Specifically, the green tea peptide composition may promote mitochondrial biosynthesis in the fat cell by increasing the expression of one or more of TFAM, NDUFA9, COX4, ATP5a, and UCP2.

In an embodiment, the green tea peptide composition may be included in an amount of 1 to 50 wt % based on the total weight of the composition for anti-obesity. Specifically, the green tea peptide composition may be included in an amount of 1 wt % or more, 3 wt % or more, 5 wt % or more, 7 wt % or more, 10 wt % or more, 12 wt % or more, 14 wt % or more, 16 wt % or more, 18 wt % or more, 20 wt % or more, 22 wt % or more, 24 wt % or more, 26 wt % or more, 28 wt % or more, 30 wt % or more, 32 wt % or more, 34 wt % or more, 36 wt % or more, 38 wt % or more, 40 wt % or more, 42 wt % or more, 44 wt % or more, 46 wt % or more or 48 wt % or more based on the total weight of the composition for anti-obesity. In addition, the green tea peptide composition may be included in an amount of 50 wt % or less, 48 wt % or less, 46 wt % or less, 44 wt % or less, 42 wt % or less, 40 wt % or less, 38 wt % or less, 36 wt % or less, 34 wt % or less, 32 wt % or less, 30 wt % or less, 28 wt % or less, 26 wt % or less, 24 wt % or less, 22 wt % or less, 20 wt % or less, 18 wt % or less, 16 wt % or less, 14 wt % or less, 12 wt % or less, 10 wt % or less, 8 wt % or less, 7 wt % or less, 5 wt % or less or 3 wt % or less based on the total weight of the composition for anti-obesity.

In an embodiment, the green tea peptide composition may be administered in an amount of 1 to 400 mg/kg/day. Specifically, the green tea peptide composition may be administered in an amount of 1 mg/kg/day or more, 5 mg/kg/day or more, 10 mg/kg/day or more, 20 mg/kg/day or more, 30 mg/kg/day or more, 40 mg/kg/day or more, 50 mg/kg/day or more, 60 mg/kg/day or more, 70 mg/kg/day or more, 80 mg/kg/day or more, 90 mg/kg/day or more, 100 mg/kg/day or more, 150 mg/kg/day or more, 200 mg/kg/day or more, 250 mg/kg/day or more, 300 mg/kg/day or more or 350 mg/kg/day or more. In addition, the green tea peptide composition may be administered in an amount of 400 mg/kg/day or less, 350 mg/kg/day or less, 300 mg/kg/day or less, 250 mg/kg/day or less, 200 mg/kg/day or less, 150 mg/kg/day or less, 100 mg/kg/day or less, 90 mg/kg/day or less, 80 mg/kg/day or less, 70 mg/kg/day or less, 60 mg/kg/day or less, 50 mg/kg/day or less, 40 mg/kg/day or less, 30 mg/kg/day or less, 20 mg/kg/day or less, 10 mg/kg/day or less or 5 mg/kg/day or less.

In an embodiment, the composition for anti-obesity may be a pharmaceutical or food composition. More specifically, the composition may be a pharmaceutical composition for anti-obesity or a health functional food composition for anti-obesity.

The formulation of the food composition is not particularly limited, but may be formulated, for example, as tablets, granules, pills, powders, liquids such as beverages, caramels, gels, bars, tea bags, and the like. In addition to the active ingredient, the food composition of each formulation can be prepared by a person of ordinary skill in the art without difficulty, depending on the formulation or the purpose of use, with ingredients conventionally used in the art properly selected and combined, and synergistic effects may occur when applied simultaneously with other ingredients.

The composition may be administered in a variety of ways, including simple ingestion, drinking, injection administration, spray administration, or squeeze administration.

The food composition according to one aspect of the present invention may be, for example, a variety of food products, such as chewing gums, caramel products, candies, ice cream, confections, and the like; a beverage product, such as a soft drink, mineral water, or alcoholic beverage; or a health functional food product, including vitamins or minerals.

The food composition according to one aspect of the present invention may include food additives in addition to the active ingredient. The food additive may be generally understood as a material that is added to, mixed with, or infiltrated into food in the manufacturing, processing, or preservation of food, which will be consumed daily and for a long period of time with the food, and thus its safety should be ensured. The food additive code, which is based on the laws of each country that regulate the manufacture and distribution of food (in Korea, the “Food Hygiene Act”), stipulates that the food additives that are guaranteed to be safe are limited in terms of ingredients or functions. In the Korea Food Additives Code (“Standards and Specifications for Food Additives” notified by Ministry of Food and Drug Safety), food additives are classified as chemical synthetics, natural additives, and mixed preparations in terms of ingredients, and these food additives are classified as sweeteners, flavoring agents, preservatives, emulsifiers, acidulants, and thickeners in terms of functions.

The sweetener is used to provide a moderate sweetness to the food product, and all the natural or synthetic sweetener may be used in the food composition according to one aspect of the present invention. Preferably, the natural sweetener is used, and examples of the natural sweeteners include corn syrup solids, honey, and sugar sweeteners such as sucrose, fructose, lactose, and maltose.

The flavoring agent is used to enhance taste or aroma, and all the natural and synthetic flavoring agents may be used. Preferably, the natural flavoring agent is used. When the natural flavoring agent is used, it may serve a nutritional purpose in addition to flavor. The natural flavoring agent may be one obtained from apples, lemons, tangerines, grapes, strawberries, peaches, and the like, or from green tea leaves, solomon's seal, bamboo leaves, cinnamon, chrysanthemum leaves, jasmine, and the like. In addition, the natural flavoring agent may be one obtained from ginseng (red ginseng), bamboo shoots, aloe vera, Ginkgo biloba, and the like. The natural flavoring agent may be a concentrate in the liquid phase or an extract in the solid phase. In some cases, the synthetic flavoring agent may be used, which may include ester, alcohol, aldehyde, terpene, and the like.

The preservatives may include calcium sorbate, sodium sorbate, potassium sorbate, calcium benzoate, sodium benzoate, potassium benzoate, and ethylenediaminetetraacetic acid (EDTA). In addition, the emulsifier may include acacia gum, carboxymethyl cellulose, xanthan gum, pectin, and the like, and the acidulant may include citric acid, malic acid, fumaric acid, adipic acid, gluconic acid, tartaric acid, ascorbic acid, acetic acid, phosphoric acid, and the like. The acidulant may be added to bring the food composition to a suitable acidity level for the purpose of inhibiting the growth of microorganisms in addition to the purpose of enhancing taste. The thickening agent may include a suspension enabler, a sedimentation agent, a gel-forming agent, a swelling agent, and the like.

The food composition according to one aspect of the present invention may include, in addition to the food additives described above, bioactive materials or minerals that are known in the art and whose stability as food additives is assured for the purpose of supplementing and enhancing functionality and nutrition.

The bioactive materials may include catechins, such as those found in green tea, vitamins such as vitamin B1, vitamin C, vitamin E, vitamin B12, tocopherols, dibenzoyl thiamine, etc., and the mineral may include calcium preparations such as calcium citrate, magnesium preparations such as magnesium stearate, iron preparations such as iron citrate, chromium chloride, potassium iodide, selenium, germanium, vanadium, zinc, and the like.

The food composition according to one aspect of the present invention may include the food additive as described above in a suitable amount to accomplish the purpose for which the food additive is added, depending on the type of product.

With respect to other food additives that may be included in the food composition according to one aspect of the present invention, the food code or food additive code of each country may be referenced.

The pharmaceutical composition according to one aspect of the present invention may be prepared as an oral formulation or a parenteral formulation, depending on the route of administration, by conventional methods known in the art, including a pharmaceutically acceptable carrier in addition to the active ingredient. Here, the route of administration may be an arbitrary suitable route, including a topical route, an oral route, an intravenous route, an intramuscular route, and direct absorption through mucosal tissue, and a combination of two or more routes may be used. An example of the combination of two or more routes is a combination of two or more formulations of drugs by route of administration, for example, one drug is administered primarily by an intravenous route and the other drug is administered secondarily by a topical route.

The pharmaceutically acceptable carrier is known in the art, depending on the route of administration and formulation, and specifically the pharmacopoeia of each country, including the Korean Pharmacopoeia may be referenced.

When the pharmaceutical composition according to one aspect of the present invention is formulated in an oral formulation, the pharmaceutical composition may be prepared in the formulation of a powder, granule, tablet, pill, sugar coated tablet, capsule, liquid, gel, syrup, suspension, wafer, or the like according to methods known in the art in combination with a suitable carrier. In this case, examples of the suitable carriers may include sugars such as lactose, glucose, sucrose, dextrose, sorbitol, mannitol, and xylitol, starches such as corn starch, potato starch, and wheat starch, celluloses, such as cellulose, methylcellulose, ethyl cellulose, sodium carboxymethylcellulose, and hydroxypropyl methylcellulose, polyvinyl pyrrolidone, water, methyl hydroxybenzoate, propyl hydroxybenzoate, magnesium stearate, mineral oils, malt, gelatin, talc, polyols, vegetable oils, ethanol, and griseoil. For formulation, suitable binders, lubricants, disintegrants, colorants, diluents, and the like may be included as necessary. The suitable binders may include starch, magnesium aluminum silicate, starch ferrite, gelatin, methylcellulose, sodium carboxymethylcellulose, polyvinylpyrrolidone, glucose, corn sweetener, sodium alginate, polyethylene glycol, wax, and the like, and the lubricants may include sodium oleate, sodium stearate, magnesium stearate, sodium benzoate, sodium acetate, sodium chloride, silica, talcum, stearic acid, magnesium and calcium salts thereof, polyethylene glycol, and the like, and the disintegrants may include methyl cellulose, agar, bentonite, xanthan gum, alginic acid, or sodium salts thereof. In addition, the diluents may include lactose, dextrose, sucrose, mannitol, sorbitol, cellulose, glycine, and the like.

When the pharmaceutical composition according to one aspect of the present invention is formulated in a parenteral formulation, the pharmaceutical composition may be formulated in the form of an injectable, transdermal, nasal inhalation, and suppository according to the method known in the art in combination with a suitable carrier. When the pharmaceutical composition is formulated as an injectable, the suitable carrier may be an aqueous isotonic solution or suspension, specifically, phosphate buffered saline (PBS) containing triethanolamine, sterile water for injection, or an isotonic solution such as 5% dextrose may be used as the carrier. When the pharmaceutical composition is formulated as a transdermal dosage form, the pharmaceutical composition may be formulated as ointments, creams, lotions, gels, topical solutions, pastes, liniments, aerosols, and the like. When the pharmaceutical composition is formulated as a nasal inhalation, the pharmaceutical composition may be formulated as the form of an aerosol spray using a suitable propellant such as dichlorofluoromethane, trichlorofluoromethane, dichlorotetrafluoroethane, carbon dioxide, and the like, and when the pharmaceutical composition is formulated as a suppository, witepsol, tween 61, polyethylene glycol, cacao butter, laurin butter, polyoxyethylene sorbitan fatty acid esters, polyoxyethylene stearates, sorbitan fatty acid esters, and the like may be used as the carrier.

The amount of application or administration of the pharmaceutical composition according to one aspect of the present invention will depend on the age, sex, weight, pathologic condition and its severity of the subject to be administered, the route of administration, or the judgment of the prescriber. The determination of the amount of administered active ingredient based on these factors falls within the skill of a person of ordinary skill in the art.

The composition for anti-obesity according to one aspect of the present invention may further include, in addition to the active ingredient, an arbitrary compound or natural extract already known in the art to be safe and to have the corresponding activity, in order to enhance or augment the anti-obesity effect or to improve the convenience of administration or ingestion through the addition of a similar activity, such as blood pressure regulating activity. These compounds or extracts include compounds or extracts listed in official documents such as the Korean Pharmacopoeia (in Korea, the “Korean Pharmacopoeia”), the Korean Health Functional Food Official Code (in Korea, the “Health Functional Food Standards and Specifications” notified by the Ministry of Food and Drug Safety), compounds or extracts that have been approved for products under the laws of each country governing the manufacture and sale of drugs (in Korea, the “Pharmaceutical Affairs Act”), and compounds or extracts whose functionality is certified under the laws of each country governing the manufacture and sale of health functional foods (in Korea, the “Health Functional Foods Act”).

For example, according to the Korean “Health Functional Foods Act”, the followings may correspond to these compounds or extracts: garcinia cambogia peel extract, conjugated linolenic acid (free fatty acid), conjugated linolenic acid (triglyceride), green tea extract, chitosan, Lactobacillus gasseri BNR17, L-carnitine tartrate, green maté extract, green coffee bean extract, sesame leaf extract, soybean germ extract, gynostemma leaf alcohol extract powder, lactoferrin (milk purified protein), lemon balm extract mixed powder, mate hydrothermal extract, complex extract of seaweed and others (Xanthigen), fermented vinegar pomegranate complex, boicha extract, mouse eye bean peptide complex, vegetable oil diglyceride, wild mango seed extract, oil containing medium chain fatty acids (MCFA), coleus forskohlii extract, chitooligosaccharides, finger root extract powder, complex extract of hibiscus and others, which are recognized for the functionality of ‘reducing body fat’, and L-glutamic acid-derived GABA-containing powder, katsuobushi oligopeptide, natto fungus culture powder, mouse eye bean peptide complex, salmon peptide, olive leaf extract, sardine peptide, casein hydrolysate, coenzyme Q10, grape seed enzymolysis extract powder, haetae oligopeptide, and the like, which are recognized for the functionality of ‘blood pressure control’, and DHA concentrated oil, globin hydrolysate, ovarian maltodextrin, bamboo leaf extract, vegetable oil diglycerides, sardine purified fish oil, purified squid oil, and the like, which are recognized for the functionality of ‘improving triglycerides in the blood’, and L-arabinose, nopal extract, cinnamon extract powder, guava leaf extract, nondigestible maltodextrin, freeze-dried silkworm powder, hemp alcohol extract, banaba leaf extract, morus alba extract, and the like, which are recognized for the functionality of ‘regulating blood sugar’, and fermentation-generated amino acid complex, hovenia dulcis fruit extract, Rhodiola rosea extract, and the like, which are recognized for the functionality of ‘improving fatigue’, and L-theanine, ashwagandha extract, whey protein hydrolysate, gynostemma leaf extract, and the like, which are recognized for the functionality of ‘anti-stress’.

In an example, the present invention may provide the following embodiments.

A first embodiment may provide a composition for anti-obesity, including a green tea peptide composition as an active ingredient.

A second embodiment may provide the composition for anti-obesity according to the first embodiment, in which the green tea peptide composition includes one or more species of green tea peptides including an amino acid sequence selected from the group consisting of SEQ ID NO: 1 to SEQ ID NO: 7.

A third embodiment may provide the composition for anti-obesity according to one or more of the first and second embodiments, in which the green tea peptide composition is obtained by fermenting a green tea protein with plant-based lactic acid bacterium.

A fourth embodiment may provide the composition for anti-obesity according to one or more of the first to third embodiments, in which the plant-based lactic acid bacterium is Lactiplantibacillus plantarum.

A fifth embodiment may provide the composition for anti-obesity according to one or more of the first to fourth embodiments, in which the green tea protein is obtained from a residue of a primary extract that is extracted from green tea with anhydrous or hydrous C1-C6 low carbon alcohol.

A sixth embodiment may provide the composition for anti-obesity according to one or more of the first to fifth embodiments, in which a concentration of an alcohol in the hydrous C1-C6 low carbon alcohol is 20 to 80% (v/v).

A seventh embodiment may provide the composition for anti-obesity according to one or more of the first to sixth embodiments, in which the hydrous C1-C6 low carbon alcohol is an ethanol aqueous solution of 20 to 80% (v/v).

An eighth embodiment may provide the composition for anti-obesity according to one or more of the first to seventh embodiments, in which the green tea protein is obtained from a residue of a secondary extract that is hydrothermally extracted from the residue of the primary extract.

A ninth embodiment may provide the composition for anti-obesity according to one or more of the first to eighth embodiments, in which the green tea peptide composition inhibits lipid synthesis in a fat cell.

A tenth embodiment may provide the composition for anti-obesity according to one or more of the first to ninth embodiments, in which the green tea peptide composition reduces an expression of one or more of SREBP1c, ACC, FAS, and SCD1.

An eleventh embodiment may provide the composition for anti-obesity according to one or more of the first and tenth embodiments, in which the green tea peptide composition promotes fat oxidation in the fat cell.

A twelfth embodiment may provide the composition for anti-obesity according to one or more of the first and eleventh embodiments, in which the green tea peptide composition increases an expression of one or more of ACO, CPT, mCAD, and PPARa.

A thirteenth embodiment may provide the composition for anti-obesity according to one or more of the first and twelfth embodiments, in which the green tea peptide composition promotes mitochondrial biosynthesis in the fat cell.

A fourteenth embodiment may provide the composition for anti-obesity according to one or more of the first to thirteenth embodiments, in which the green tea peptide composition increases an expression of one or more of TFAM, NDUFA9, COX4, ATP5a, and UCP2.

A fifteenth embodiment may provide the composition for anti-obesity according to one or more of the first to fourteenth embodiments, in which the green tea peptide composition is included in an amount of 1 to 50 wt % based on the total weight of the composition.

A sixteenth embodiment may provide the composition for anti-obesity according to one or more of the first to fifteenth embodiments, in which the green tea peptide composition is administered in an amount of 1 to 400 mg/kg/day.

A seventeenth embodiment may provide the composition for anti-obesity according to one or more of the first to sixteenth embodiments, in which the composition is a pharmaceutical composition or a health functional food composition.

Hereinafter, the content of the present invention will be described in more detail through examples and test examples. However, these embodiments and examples are presented for the purpose of understanding the content of the present invention, and the scope of the present invention is not limited to these embodiments and examples, and modifications, substitutions, and insertions known in the art can be made, which are also included in the scope of the present invention.

### [Example 1] Preparation of Green Tea Peptide Composition (GTP)

The green tea of 50 kg (Camelia sinensis, Agricultural Corporation Osulloc Farm) was added to a 1-ton extraction tank, ethanol of 50% (v/v) was added at 15 times, and then extracted (primary extraction) at 70° C. for 2 hours and filtered to remove catechins and obtain a residue of primary extract of the green tea. Purified water was added to the residual solids of the primary extract of the green tea at a ratio of 15 times, which was then extracted (secondary extraction) at 90° C. for 3 hours and filtered to remove water-soluble polysaccharides, etc. to obtain the residue of the secondary extract of the green tea. A 2% (w/w) aqueous solution of NaOH (98%, Youngjin Co., Ltd.) was added to the residual solids of the secondary extract of the obtained green tea at a ratio of 10 times, which was extracted (alkaline extraction) at 70° C. for three hours and filtered to obtain the filtrate. The obtained filtrate was cooled to room temperature and 35% (w/w) hydrochloric acid (Daejung Chemicals) was added to adjust the pH to 3.5 to 4.5 or less. The supernatant was removed and the precipitate was washed three to seven times with purified water, and then the precipitate was spray dried using an Ohkawara OC-16 spray dryer (inlet 220° C., outlet 90° C.)to obtain the green tea protein with a crude protein content of 50% (w/w) or more. Lactiplantibacillus plantarum APsulloc 331261 culture medium (containing purified water, vitamin solution, amino acid solution, and mineral solution) containing the green tea protein of 1% (w/w) was added to an anaerobic fermenter and incubated at pH 6.8 and 37° C. for 48 hours, and then the culture medium was centrifuged (Labogene 1580R (Serial No. KLG4226180220023) at 4° C., 10,000 g for 20 minutes to obtain a supernatant. The obtained supernatant was concentrated in a dry oven at 70° C., and the concentrate was centrifuged (Hitachi centrifuge CS150NX) at 4° C., 100,000 g for one hour to obtain a supernatant. The obtained supernatant was subjected to membrane filtration (pore size 0.22 μm) to obtain a filtrate, and the obtained filtrate was freeze-dried to obtain the green tea peptide composition (green tea peptide; GTP) (green tea peptide content 14% (w/w)).

Meanwhile, the sequence of the green tea peptide contained in the green tea peptide composition was analyzed in the following steps:
1) Centrifuging the Lactiplantibacillus plantarum APsulloc 331261 culture medium containing the green tea protein to separate the supernatant, and obtaining only the peptide fractionated in the low molecular weight peptide fraction by membrane filtration and size exclusion chromatography;
2) freeze-drying and de-salting the peptide obtained from 1), then dissolving the peptide in 0.1% (w/w) formic acid and analyzing the peptide by LC-MS/MS, In this case, LC-MS/MS analysis was performed on 3 μg of sample (based on protein quantification), and the equipment and analysis conditions used were as follows;

TABLE 1

| LC equipment | UltiMate 3000 RSLC nano system |
|---|---|
| MS equipment | Q-Exactive Orbitrap HF-X mass spectrometer |
| | (Thermo Fisher Scientific) |
| Column | (trap) Internal diameter: 75 μm × 2 cm, |
| | packed with Acclaim |
| | PepMap 100 C18, 3 μm |
| | (analytical) Internal diameter: |
| | 75 μm × 50 cm, packed with PepMap |
| | RSLC C18, 2 μm |
| Mobile phase | (solvent A) 0.1% (w/w) formic acid (FA) in water |
| | (solvent B) 0.1% (w/w) FA in acetonitrile |
| Mode | Positive ion mode |
| Collision energy | 27% |

3) Searching for peptide sequences using the green tea (Camelia sinensis [UniProt Proteome ID: UP000327468]) and lactic acid bacterium (L. plantarum DSM 20174 [NCBI accession: GCA_014131735.1], L plantarum APsulloc 331261) proteome sequence database for the spectra files obtained through LC-MS/MS analysis in 2). In this case, the analysis conditions used were as follows.

TABLE 2

| | |
|---|---|
| Search engine | MS-GF+ |
| Modifications | (fixed) None |
| | (variable) oxidation (+15.99) of |
| | methionine & acetylation (+42.01) of |
| | the peptide N-terminal |
| m/z tolerance | Precursor: ±10 ppm |
| | Fragment: ±20 ppm |
| False discovery | 1% |
| rate cut-off | |

The green tea peptide sequences according to an embodiment of the present invention analyzed by the above steps is as follows.

TABLE 3

| | |
|---|---|
| AYKRRKGKFA | SEQ ID NO: 1 |
| FFFFFFFFFFFFFFFYL | SEQ ID NO: 2 |
| ISKIWNSEVPETEVKNEAESP | SEQ ID NO: 3 |
| PFFCEKMMETN | SEQ ID NO: 4 |
| RFLHERMAYYH | SEQ ID NO: 5 |
| RNLNRLQRLLSMKQEYSPRNHLGSRWREY | SEQ ID NO: 6 |
| TTSSRKKEKPRRFWNNHEEVFLITTK | SEQ ID NO: 7 |

[Experimental example 1] Confirmation of lipid metabolism-related gene expression changes in fat cell by treatment with green tea peptide composition (GTP) by concentration Obesity refers to a phenomenon in which surplus nutrition (energy) is excessively stored in a fat cell in the form of fat. In other words, the primary indicator for improving obesity is the inhibition of lipid accumulation in the fat cell (body fat reduction). To inhibit lipid accumulation in the fat cell, lipid synthesis in the fat cell needs to be inhibited and fat consumption (lipolysis, fat oxidation, etc.) needs to be promoted. In this regard, to confirm whether the green tea peptide may inhibit fatty acid synthesis in the fat cell and promote fat burning, 3T3-L1 preadipocytes purchased from ATCC were differentiated into a fat cell. For the fat cell differentiation, 3T3-L1 were cultured in DMEM (Sigma Aldrich)+10% bovine calf serum (Gibco) culture medium until the 3T3-L1 were 100% confluent and then allowed to grow for two more days to induce a complete adhesion between cells. Differentiation into the fat cell was initiated by 48 hours treatment in DMEM+10% fetal bovine serum (FBS; Gibco) culture medium supplemented with dexamethasone (1 μM; Sigma Aldrich), insulin (10 μg/ml; Sigma Aldrich), and 3-isobutyl-1-methylxanthine (0.5 mM; Sigma Aldrich). After 48 hours, the cell was further cultured with DMEM+10% FBS+10 μl g/ml insulin culture medium to induce complete fat cell differentiation, and an occasion in which 80% or more of the total cells had differentiated from preadipocytes to the fat cell, with the cell shape changing to a round shape and containing adipocytes inside, was selected as an occasion in which the differentiation was complete. Antibiotics (penicillin/streptomycin 100 U each, 100 mg/ml; Gibco) were added to the culture medium to prevent cell contamination by microorganisms in the process of culturing cells.

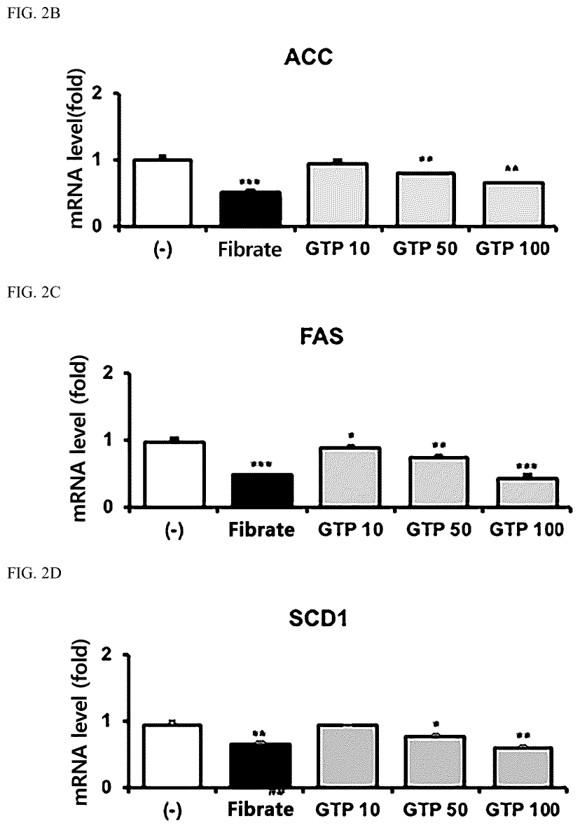

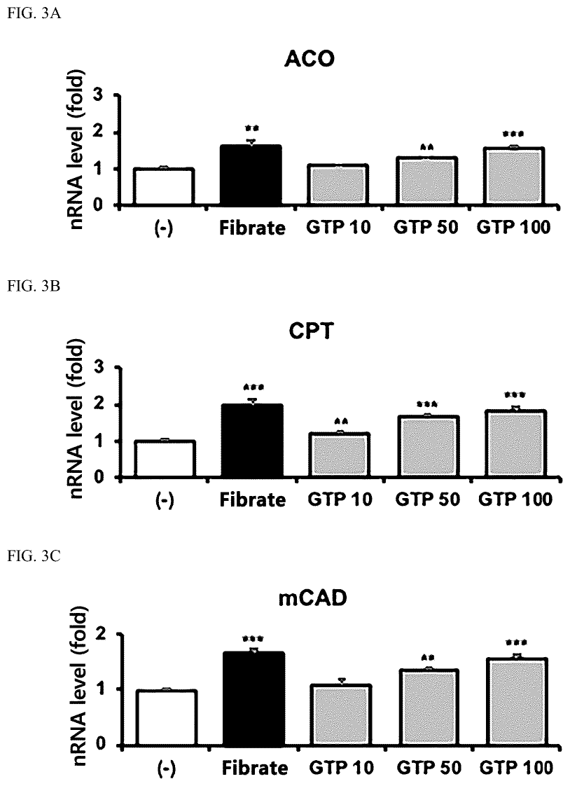

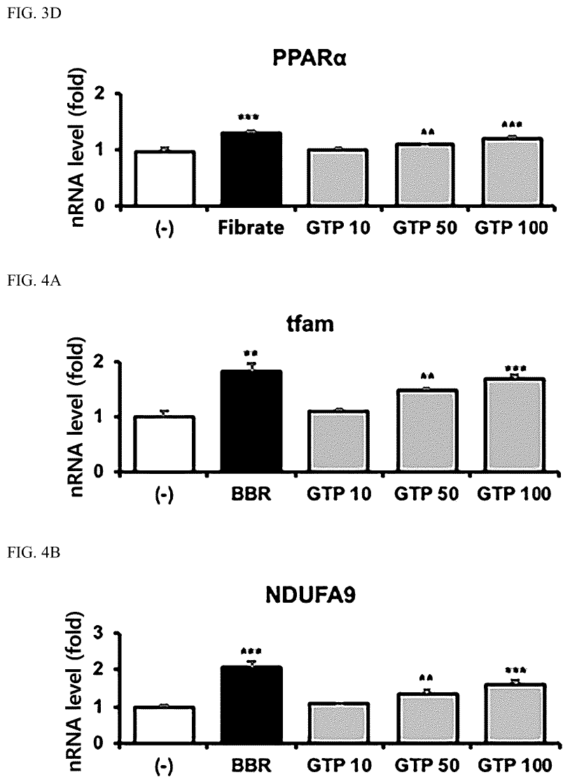

The differentiated fat cell was then treated with the green tea peptide composition (GTP) of Example 1 at various concentrations (10, 50, 100 μ g/ml) for 24 hours, and RNA was extracted from the fat cell using the TaKaRa MiniBEST Universal RNA Extraction Kit (Takara Bio). The cDNA was synthesized using the RevertAid 1st-strand cDNA Synthesis Kit (Thermo Fisher Scientific), and the changes in expression of fat metabolism-related genes were observed using a CFX96 thermocycler (Bio-Rad). For the positive control, fenofibrate (100 μM; Sigma Aldrich), a drug that promotes fat oxidation and inhibits fat synthesis through PPAR a activation, was used. The results are shown in FIGS. 2A to 2D and FIGS. 3A to 3D.

As illustrated in FIGS. 2A to 2D, it could be confirmed that the fat cell treated with the green tea peptide composition (GTP) according to Example 1 showed a concentration-dependent decrease in the expression of fat synthesis-related genes (SREBP1c, ACC, FAS, SCD1), and in particular, it could be confirmed that the expression of fat synthesis-related genes was reduced to a level equivalent to or better than the positive control, fenoFibrate.

Further, from the results in FIGS. 3A to 3D, it could be confirmed that the expression of fat oxidation-related genes (ACO, CPT, mCAD, PPARa) increased in a concentration-dependent manner in the fat cell treated with the green tea peptide composition according to Example 1, and in particular, it could be confirmed that the expression of fat oxidation-related genes increased to a level almost equivalent to the positive control fenoFibrate.

From the above, it can be seen that the green tea peptide composition according to one aspect of the present invention will exhibit an excellent anti-obesity effect by inhibiting fat synthesis in the fat cell and promoting fat oxidation.

[Experimental example 2] Confirmation of increase in mitochondria in fat cell by treatment with green tea peptide composition (GTP) by concentration

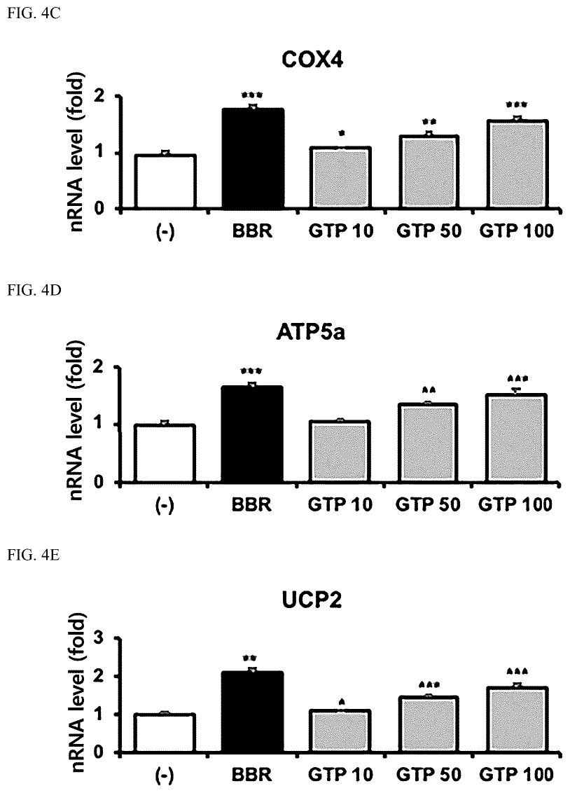

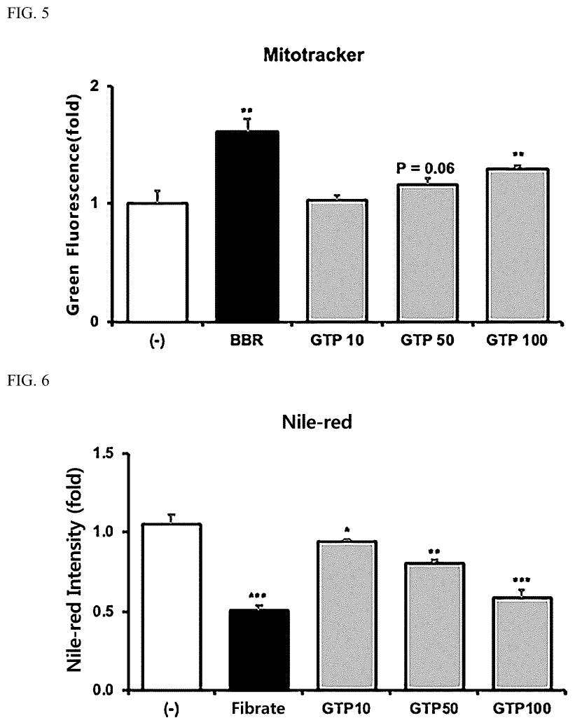

From the results of Example 1 above, it is expected that the green tea peptide composition according to one aspect of the present invention will inhibit fat synthesis and promote fatty acid oxidation. However, the processes of fatty acid breakdown and ATP production through the Krebs cycle and electron transport system inevitably lead to oxidative stress. Sufficient amounts of mitochondria and uncoupling proteins can assist in inhibiting the occurrence of excessive oxidative stress and allowing more fat to be safely burned. In this regard, to investigate whether the green tea peptide does not simply affect fat oxidation but also induces both the mitochondrial biosynthesis and expression of uncoupling proteins, the expression of mitochondrial biosynthesis-related genes was further confirmed using the same method as in Example 1 above, and the results were shown in FIG. 4A to FIG. 4E. In addition, the results of confirming whether mitochondria are increased in the fat cell are shown in FIG. 5.

As illustrated in FIGS. 4A to 4E, it could be confirmed that the green tea peptide composition (GTP) according to Example 1 also increased the expression of mitochondrial and electron transport system component genes (tfam, NDUFA9, COX4, ATP5a, and UCP2) to facilitate fatty acid oxidation and energy production.

In addition, from the results in FIG. 5, it could be confirmed that the green tea peptide composition (GTP) according to Example 1 significantly increased mitochondria in the fat cell.

These results indicate that the green tea peptide composition according to one aspect of the present invention may help to induce the body to burn more fat for cellular maintenance by converting (consuming) protons stored for ATP production into heat, as well as reduce the probability of occurrence of oxidative stress due to excessive accumulation of protons beyond the capacity of the electron transport system.

[Experimental example 3] Confirmation of effect of green tea peptide composition (GTP) on inhibition of lipid accumulation in fat cell

It was confirmed that the green tea peptide can inhibit fatty acid synthesis and promote fatty acid oxidation through Experimental example 1 above. In this regard, to confirm whether the green tea peptide can actually reduce the amount of fat accumulated in the fat cell, the differentiated 3T3-L1 fat cell was treated with the green tea peptide composition (GTP) of Example 1 above at various concentrations (10, 50, and 100 μg/ml) for 72 hours. Then, the cell was fixed in 10% formaldehyde solution for 5 minutes and stained using 300 nM of Nile-red (Sigma Aldrich) solution. The amount of fat accumulated was quantified using a Tecan Infinite M200 Multiplate Reader equipment (Tecan Trading AG; excitation 495 nm, emission 585 nm). The results are shown in FIG. 6.

As illustrated in FIG. 6, it could be confirmed that the amount of triglycerides accumulated in the fat cell was significantly reduced upon treatment with the green tea peptide composition according to one aspect of the present invention.

[Experimental example 4] Confirmation of effect of inhibiting inflammatory response in fat cell by green tea peptide composition (GTP)

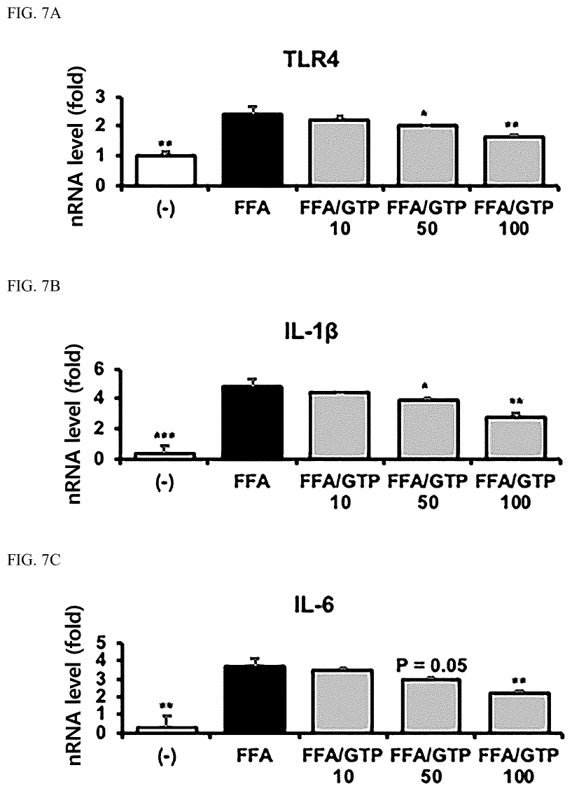

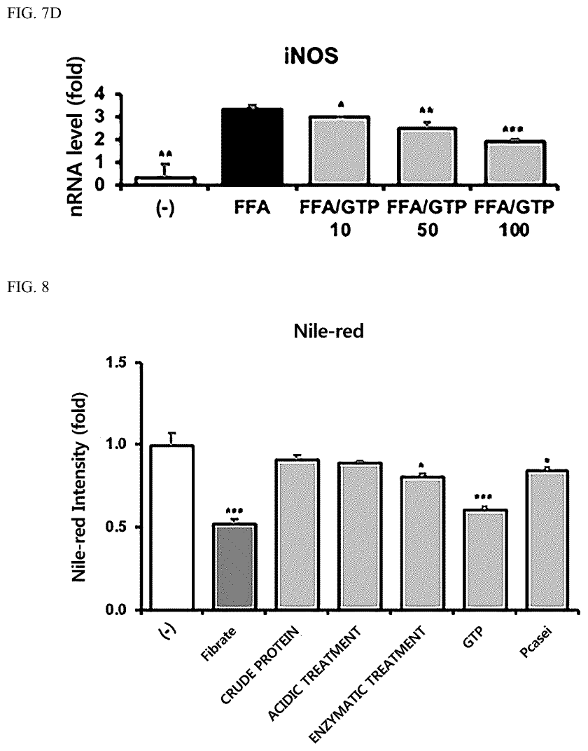

The inflammatory response is the first line of defense against foreign contaminants and is mediated by TLR4, which can detect bacterial cell wall components (LPS). However, TLR4 recognizes not only LPS but also free fatty acids (FFAs) to mediate the inflammatory response, and the phenomenon of triggering an inflammatory response on its own without the presence of external substances is known as a chronic inflammatory response. Since the chronic inflammatory response disrupts the insulin signaling system, leading to various metabolic complications such as insulin resistance, diabetes, and hyperlipidemia, improving the chronic inflammatory response within the fat cell and furthermore in the body is very important to prevent metabolic diseases caused by obesity. In this regard, to confirm whether the green tea peptide composition according to one aspect of the present invention can ameliorate the free fatty acid-mediated inflammatory response induced by obesity, the differentiated fat cell was pretreated with the green tea peptide composition at concentrations of 10, 50, and 100 μg/ml for 24 hours and then treated with FFA (0.5 mM, Sigma Aldrich) for 12 hours to induce an inflammatory response. After RNA isolation and cDNA synthesis were performed using the same method as in Example 1 above, the expression of inflammatory response-related genes was observed by Q-PCR, and the results are shown in FIGS. 7A to 7D.

From the results in FIGS. 7A to 7D, it could be confirmed that the green tea peptide composition (GTP) according to Example 1 significantly reduced the expression of inflammatory response-related genes (TLR4, IL-1B, IL-6, and iNOS) in the fat cell, effectively improving the inflammatory response occurring in the fat cell.

[Experimental example 5] Confirmation of effect of inhibiting lipid accumulation in fat cell by green tea peptide composition (GTP) according to different preparation methods

To compare the anti-obesity efficacy of a green tea peptide according to a method of preparation, i) green tea crude protein obtained during the process of preparing the green tea peptide composition of Example 1 above, ii) a supernatant (green tea protein acid-treated fraction) obtained by preparing the green tea protein obtained during the process of preparing the green tea peptide composition of Example 1 above at a concentration of 1:40 (w/v) using purified water, followed by the addition of 35% (w/w) hydrochloric acid to adjust to pH 5, and then hydrolyzing the mixture at 37° C. for 6 hours, followed by centrifugation at 4° C., 10,000 g (Labogene 1580R (Serial No. KLG4226180220023), iii) a supernatant (a green tea proteolytic enzyme-treated fraction) obtained by preparing the green tea protein obtained during the process of preparing the green tea peptide composition of Example 1 above at a concentration of 1:40 (w/v) using 0.1 M sodium phosphate buffer, pH 8, followed by the addition of the proteolytic enzyme bromelain at 1% (w/v) relative to the green tea protein, and then hydrolyzing the mixture at 45° C., pH 6.2 for 24 hours, and heating the mixture at 90° ° C. for 30 minutes, followed by centrifugation at 4° C., 10,000 g (Labogene 1580R (Serial No. KLG4226180220023)), and iv) a peptide composition prepared in the same manner as in Example 1 above, except that Lacticaseibacillus paracasei (KCTC 3510 (ATCC Strain No.: ATCC 25302). Korean Collection for Type Cultures (KCTC)), which is an animal lactic acid bacterium, was used instead of Lactiplantibacillus plantarum APsulloc 331261, were treated together to compare the anti-obesity efficacy. The specific experimental method was performed as in Experimental example 3 above, except that the green tea crude protein, the green tea protein acid-treated fraction, the green tea proteolytic enzyme (bromelain)-treated fraction, and the peptide composition fermented by Lacticaseibacillus paracasei (L. paracasei), which is an animal lactic acid bacterium, and the green tea peptide composition according to Example 1 were each treated at a concentration of 100 μg/ml for 72 hours. The results are shown in FIG. 8.

From the results in FIG. 8, it could be confirmed that the green tea crude protein, acid breakdown peptide did not exhibit significant lipid accumulation inhibition efficacy, the enzyme breakdown peptide and the animal lactic acid bacterium fermented peptide composition exhibited very marginal efficacy, while only the green tea peptide composition according to an embodiment of the present invention exhibited the highest lipid accumulation inhibition activity, almost equivalent to the positive control.

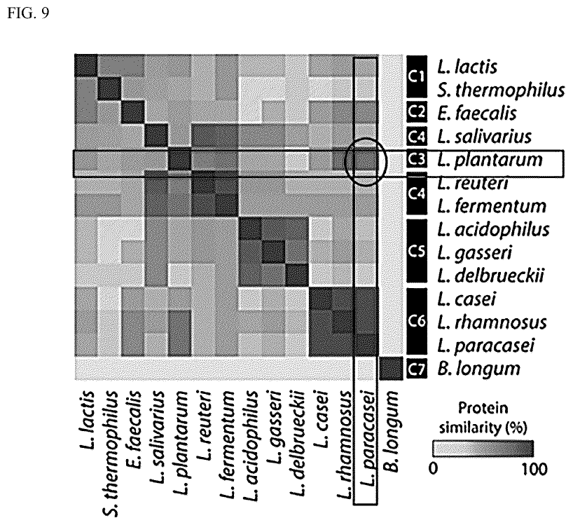

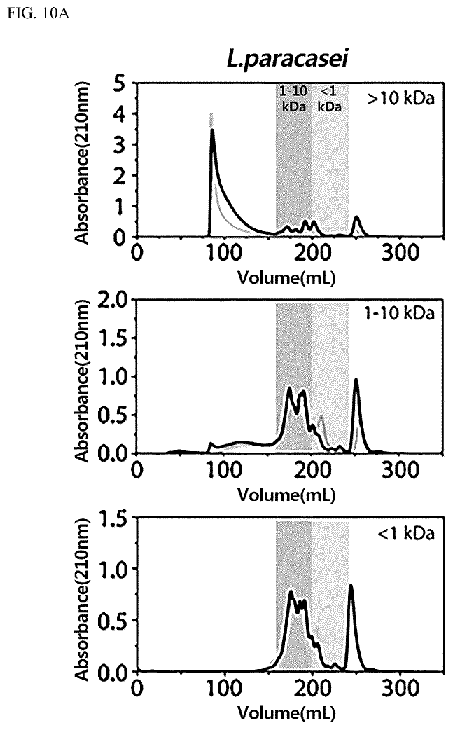

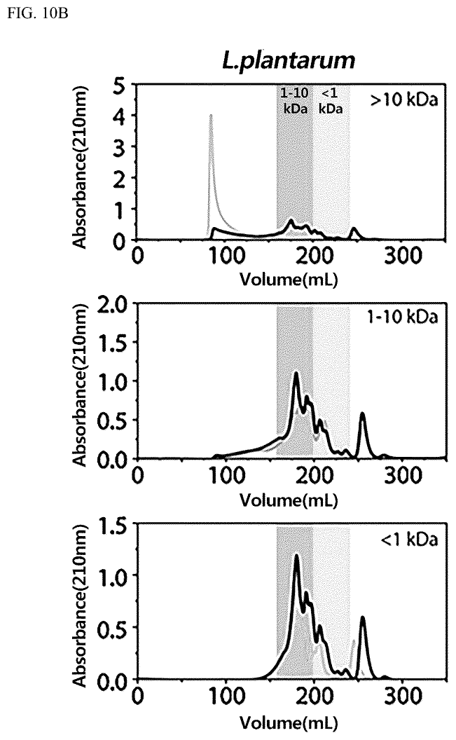

Meanwhile, FIG. 9 illustrates a result of analyzing the similarity between Lactiplantibacillus plantarum and Lacticaseibacillus paracasei (L. paracasei). Similarity analysis was calculated by downloading the genome information (genbank) for each strain from NCBI (ncbi.nlm.nih.gov/genbank/), retaining only the annotated conserved protein genetic information, discarding the rest thereof, followed by calculation as a percentage of the total number. In this case, the R program was used to calculate the percentage of conserved proteins (POCP) (code source: github.com/hoelzer/pocp.git), and the analysis conditions were as follows:
E-value=1×e⁻⁵
Sequence identity=0.4
Alignment length=0.5.

Among these, two genomes were categorized into the same cluster when they exhibited 50% or more of similarity, and Lactiplantibacillus plantarum and Lacticaseibacillus paracasei (L. paracasei) were found to be very similar with 46.84% of similarity. As described above, it could be confirmed that the green tea peptide composition (PCasei) obtained by fermentation with Lacticaseibacillus paracasei (L. paracasei), which is an animal lactic acid bacterium very similar to the Lactiplantibacillus plantarum used in Example 1, did not exhibit efficacy for inhibiting lipid accumulation in the fat cell, i.e., anti-obesity efficacy. Therefore, it can be seen that the anti-obesity efficacy of the green tea peptide composition according to an embodiment of the present invention is obtained through fermentation using Lactiplantibacillus plantarum, which is a plant-based lactic acid bacterium.

[Experimental example 6] Confirmation of anti-obesity effect of green tea peptide (GTP) according to molecular weight

Sequence analysis of the peptide isolated and purified from the green tea peptide composition prepared in Example 1 above resulted in the identification of a peptide having an amino acid sequence of SEQ ID NO:s 1 to 7.

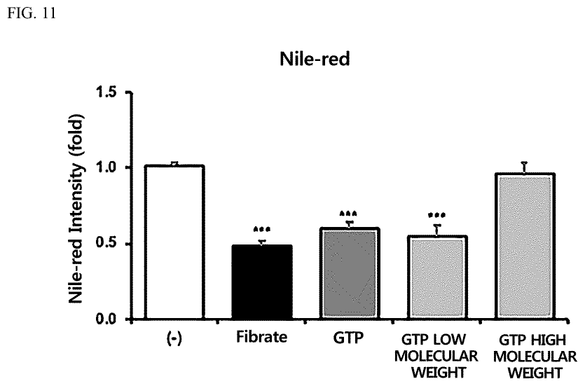

FIGS. 10A and 10B illustrate results of analyzing a molecular weight of a green tea proteolytic product by each of Lacticaseibacillus paracasei (L. paracasei) and Lactiplantibacillus plantarum. Specifically, the molecular weight analysis was performed by size exclusion chromatography using the fast protein liquid chromatography (FPLC), and the specific conditions for the FPLC performing equipment and buffer are as follows:

TABLE 4

| | |
|---|---|
| Equipment | BioLogic Duoflow |
| | Chromatography System |
| Column | Superdex 30 |
| | pg (200 ml) |
| Buffer | 20 mM Tris-HCl, |
| | pH 7.0 |
| Flow rate | 1.5 |
| | ml/min |
| Pressure | 7-9 |
| | psi |

| | | |
|---|---|---|
| Sample amount | High molecular weight fraction | 100 mg |
| (concentration) | (>10 kDa) | (50 mg/ml) |
| | Low molecular weight fraction | 100 mg |
| | (≤10 kDa) | (50 mg/ml) |
| | Green tea peptide | 100 mg |
| | composition(GTP) | (50 mg/ml) |

As illustrated in FIGS. 10A and 10B, it could be confirmed that the green tea peptide composition obtained by fermentation with Lactiplantibacillus plantarum includes mostly small molecular peptides of 10 kDa or less, unlike the green tea peptide (PCasei) fermented with Lacticaseibacillus paracasei (L. paracasei).

Accordingly, the green tea peptide composition prepared in Example 1 above was fractionated into a low molecular weight fraction and a high molecular weight fraction with reference to a molecular weight of 10 kDa using a dialysis kit (Sigma Aldrich), and each sample was treated with the concentrations listed in Table 4 above, and the efficacy of inhibiting lipid accumulation in the fat cell for each sample was compared using the same method as in Example 3 above. The results are shown in FIG. 11.

As illustrated in FIG. 11, it could be confirmed that the low molecular weight fraction containing a large amount of the novel peptide produced by Lactiplantibacillus plantarum fermentation exhibited the excellent efficacy of inhibiting lipid accumulation, while the high molecular weight fraction containing no novel peptide exhibited no efficacy of inhibiting lipid accumulation. Therefore, it can be seen that the anti-obesity efficacy of the green tea peptide composition according to an embodiment of the present invention is achieved by a novel peptide that has been broken down by Lactiplantibacillus plantarum, which is a fermentative lactic acid bacterium.

### Accession Number

Name of depository authority: Korea Culture Center of Microorganisms (overseas)
Accession Number: KCCM11179P
Accession Date: 20110328

What is claimed is:

1. A method of preventing, ameliorating, or treating obesity, the method comprising: administering an active amount of a green tea peptide composition to a subject in need thereof.

2. The method of claim 1, wherein the green tea peptide composition includes one or more species of green tea peptides including an amino acid sequence selected from the group consisting of SEQ ID NO: 1 to SEQ ID NO: 7.

3. The method of claim 1, wherein the green tea peptide composition is obtained by fermenting a green tea protein with a plant-based lactic acid bacterium.

4. The method of claim 3, wherein the plant-based lactic acid bacterium is Lactiplantibacillus plantarum.

5. The method of claim 3, wherein the green tea protein is obtained from a residue of a primary extract that is extracted from green tea with anhydrous or hydrous C1-C6 low carbon alcohol.

6. The method of claim 5, wherein a concentration of alcohol in the hydrous C1-C6 low carbon alcohol is 20 to 80% (v/v).

7. The method of claim 6, wherein the hydrous C1-C6 low carbon alcohol is a 20 to 80% (v/v) ethanol aqueous solution.

8. The method of claim 5, wherein the green tea protein is obtained from a residue of a secondary extract which is hydrothermally extracted from the residue of the primary extract.

9. The method of claim 1, wherein the green tea peptide composition inhibits lipid synthesis in a fat cell.

10. The method of claim 9, wherein the green tea peptide composition reduces an expression of one or more of SREBP1c, ACC, FAS, and SCD1.

11. The method of claim 1, wherein the green tea peptide composition promotes fat oxidation in a fat cell.

12. The method of claim 11, wherein the green tea peptide composition increases an expression of one or more of ACO, CPT, mCAD, and PPARa.

13. The method of claim 1, wherein the green tea peptide composition promotes mitochondrial biosynthesis in a fat cell.

14. The method of claim 13, wherein the green tea peptide composition increases an expression of one or more of TFAM, NDUFA9, COX4, ATP5a, and UCP2.

15. The method of claim 1, wherein the green tea peptide composition is formulated as a composition for anti-obesity, and the green tea peptide composition is contained in an amount of 1 to 50 wt % based on the total weight of the composition for anti-obesity.

16. The method of claim 1, wherein the green tea peptide composition is administered in an amount of 1 to 400 mg/kg/day.

17. The method of claim 1, wherein the green tea peptide composition is formulated as a composition for anti-obesity, and the composition for anti-obesity is a pharmaceutical or health functional food composition.


SEQUENCE LISTING (7 sequences)

SEQ ID NO: 1 - protein, 10 aa, unidentified
AYKRRKGKFA

SEQ ID NO: 2 - protein, 17 aa, unidentified
FFFFFFFFFFFFFFFYL

SEQ ID NO: 3 - protein, 21 aa, unidentified
ISKIWNSEVPETEVKNEAESP

SEQ ID NO: 4 - protein, 11 aa, unidentified
PFFCEKMMETN

SEQ ID NO: 5 - protein, 11 aa, unidentified
RFLHERMAYYH

SEQ ID NO: 6 - protein, 29 aa, unidentified
RNLNRLQRLLSMKQEYSPRNHLGSRWREY

SEQ ID NO: 7 - protein, 26 aa, unidentified
TTSSRKKEKPRRFWNNHEEVFLITTK